(19) United States
(12) Patent Application Publication
(10) Pub. No.: US 2010/0180667 A1
(43) Pub. Date: Jul. 22, 2010

(54) METHODS AND APPARATUSES FOR DETECTING ODORS

(76) Inventors: Gregory Bender, Montreal (CA); Francois Giasson, Varennes (CA); Christophe Guy, Montreal (CA); Thierry Page, Montreal (CA)

Correspondence Address: BERESKIN AND PARR LLP/S.E.N.C.R.L., s.r.l., 40 KING STREET WEST, BOX 401, TORONTO, ON, M5H 3Y2, CA

(21) Appl. No.: 12/601,401

(22) PCT Filed: Apr. 15, 2008
(86) PCT No.: PCT/CA2008/000706

§ 371 Date: Dec. 3, 2009

(30) Foreign Application Priority Data
May 24, 2007 (US) 60940011

(51) Int. Cl. G01N 33/00

(52) U.S. Cl. 73/23.34

(57) ABSTRACT

There is provided a method for detecting at least one odor in a gas sample, the method comprising at least partially reducing an amount of water present in the gas sample; and detecting the presence or absence of at least one odor in the sample. There is also provided a method for reducing losses of sensitivity of at least one gas sensor adapted to detect and/or measure at least one odor in a gas sample, the method comprising at least partially reducing an amount of water present in the gas sample before contacting the sample with the at least one sensor. Apparatuses for detecting and/or measuring odors in a gas sample are also provided.

### FIELD OF THE INVENTION

The present document relates to the field of odor detection and measurement. In particular, it relates to methods and apparatuses for detecting and/or measuring odors. It also relates to a method for reducing losses of sensitivity of gas sensors.

### BACKGROUND OF THE INVENTION

Within the human genome, there is 1 gene for hearing, 3 genes for vision, 12 genes for tasting, and 1,000 genes for smelling. The human nose contains approximately fifty million neuro-receptors connected to ten thousand primary neurons. The latter are in contact with a second layer of neurons linked with the olfactory bulb in the cerebral cortex, which is where odors are recognized. In electronic noses, the neuro-receptors are replaced by a sensor matrix. The interactions between the different gas molecules and the sensors alter certain physical properties of the latter. The overall set of sensor matrix signals yields the “olfactory signature” or “odor pattern” characteristic of a given odor and odor concentration. In the case of the electronic noses, the two neuron layers and the cerebral cortex are replaced by an algorithmic odor recognition and quantification element. The network of artificial neurons is a common solution of this mathematical problem. It is the resemblance of the device with the human olfactory system that led to its being named an “electronic nose”.

An odor is a quality of at least one chemical compound that stimulates the olfactory organ resulting in a sensation. Odor can be defined or quantified by various metrics such as the odor concentration, the odor intensity, the odor character, the odor persistence or the odor hedonic tone.

Odor concentration at the perception threshold is by definition 1 o.u./m³ (odor unit per cubic meter). Odor concentration is expressed as multiples of the perception threshold. By definition [2], the odor unit is the quantity of odorous substance that, evaporated in 1 m³ of odorless neutral gas (CNTP), triggers a physiological odor detection response in 50% of the population. The odor concentration of an odorous gas sample is determined by presenting that sample to a human panel, causing the concentration to vary due to dilution with a neutral gas in order to determine the dilution factor at the perception threshold of 50% of the panel. At that level of dilution the odor concentration, by definition, is 1 o.u./m³. The EN 13725 standard enables, among other things, the determination of the concentration of an odor by means of dynamic olfactometry; since the samples presented to the panelists are not to undergo any pre-treatment, no method for drying the odorous air is used, and the dilution air itself is dry.

The passage from an olfactory signature (the set of sensor matrix responses to an odor of known composition and concentration) to the characterization (recognition and quantification) of the odor is affected by means of a mathematical model. After prior training, the mathematical model will thus correlate an odor (nature and concentration) with its olfactory signature. The mathematical model may take into account parameters other than the sensor responses; for instance, humidity, temperature, air flow or measurement chamber pressure.

There are today various electronic nose (or electronic sensor) technologies (see an example in FIG. 2) to meet the requirements of different industry sectors. The following are among the applications of electronic noses: quality control, environmental monitoring, research and development, the military and security sectors, and the health sector. Electronic noses make it possible to measure odors objectively, precisely, repeatably and continuously.

Different sensor technologies are used for electronic noses, such as MOS (Metal-Oxide Semiconductor), QMB (Quartz Microbalance), IRS (Infra-Red Sensor), CPS (Conducting Polymer Sensor), SAW (Surface Acoustic Wave), OFS (Optical Fiber Sensor), and others. These sensor types have different sensitivity, selectivity, robustness and service life characteristics. The choice and combination of technologies depends primarily on the type of application [1].

As previously indicated, there are several major sensor families that can be used in electronic noses. Odorous molecule recognition and quantification is made indirectly by measuring changes in some physical properties of the sensors, such as electrical conductivity and the resonance frequency.

The MOS (Metal-Oxide Semiconductor) sensor family is widely used for reasons of low cost, sensitivity, broad detection spectrum and ease of use. The metal oxides used for this type of sensor (Metal-Oxide Semiconductor) are primarily tin, zinc or iron oxides, all of them are n-type intrinsic semiconductors. When heated to temperatures between 200 and 400 degrees Celsius, these semiconductors react primarily to Volatile Organic Compounds (VOCs), hydrocarbons and sulphur and nitrogen by increasing the electrical conductivity of the conducting band. The reference electrical conductivity is dictated by the adsorption of oxygen molecules on the surface coated with metal oxide. The change in electrical conductivity at the sensor surface is therefore caused by a gain or loss of electrons according to the number of oxygen molecules reacting with the gas present. In the case of tin oxide (SnO₂) sensors, there will be a gain of electrons (reducing gas) or a loss of electrons (oxidizing gas) in the conducting band. This shows that in the presence of an oxidant gas, such as NO₂, the conducting band of an n-type conductor will tend to diminish, while in the presence of a reducing gas, such as methane, the conducting band will tend to increase.

However, one of the main drawbacks [1] of the majority of chemical sensors used for measuring odor is their sensitivity to water molecules. The effect of humidity on SnO₂-type sensor response is not yet fully understood. It would seem that there are hydroxyl groups formed at the oxide surface and that they are at equilibrium with the water vapor, in accordance to the various vapour pressures. This effect would tend to alter the response of sensors by reducing their sensitivity. Various solutions have been proposed so far in order to deal with this problem. For example, the addition of various doping additives has been used [6].

### SUMMARY OF THE INVENTION

According to one aspect there is provided a method for detecting at least one odor in a gas sample, the method comprising:
at least partially reducing an amount of water present in the gas sample; and
detecting the presence or absence of at least one odor in the sample.

According to another aspect there is provided a method for reducing losses of sensitivity of at least one gas sensor adapted to detect and/or measure at least one odor in a gas sample, the method comprising at least partially reducing an amount of water present in the gas sample before contacting the sample with the at least one sensor.

According to another aspect there is provided an apparatus for detecting and/or measuring odors in a gas sample, the apparatus comprising:
means for at least partially reducing an amount of water present in the gas sample; and
at least one gas sensor adapted to detect and/or measure odors, the at least one gas sensor being in fluid flow communication with the means for at least partially reducing an amount of water present in the gas sample and being disposed downstream of the latter.

According to another aspect there is provided in an apparatus for detecting and/or measuring at least one odor in a gas sample comprising at least one gas sensor, the improvement wherein the apparatus comprises means for at least partially reducing an amount of water present in the gas sample, disposed upstream of the at least one gas sensor.

According to another aspect there is provided in an apparatus for detecting and/or measuring at least one odor in a gas sample comprising at least one metal oxide semiconductor gas sensor, the improvement wherein the apparatus comprises means for at least partially reducing an amount of water present in the gas sample, disposed upstream of the at least one gas sensor.

Water can be mainly present in the gas sample as water vapor. For example, at least 10%, 20%, 30%, 40%, 50%, 55%, 60%, 70%, or 75% of water present in the gas sample can be removed by using the previously mentioned methods and apparatuses. Alternatively, about 10 to about 75% of water can be removed. Water can be at least partially removed from the gas sample by means of a membrane adapted to be at least substantially permeable to water and at least substantially impermeable to the at least one odor. The membrane can be a hollow fiber membrane comprising at least one hollow fiber into which the gas sample is passed through. The gas sample can be passed through the membrane so as to least partially reduce the amount of water present therein so as to obtain a gas sample having a reduced content of water as compared to the gas sample before passing through the membrane. The gas sample having a reduced content of water is then contacted with the at least one gas sensor so as to detect the presence or absence of at least one odor. A purge gas is contacted with an exterior wall of the at least one hollow fiber so as to cause water to exit the membrane. Alternatively, the gas sample can be passed through the membrane so as to least partially reduce the amount of water present therein so as to obtain a gas sample having a reduced content of water as compared to the gas sample before passing through the membrane. The gas sample having a reduced content of water is then contacted with at least one gas sensor so as to detect the presence or absence of the at least one odor, and the gas sample having a reduced content of water is then contacted with an exterior wall of the at least one hollow fiber so as to cause water to exit the membrane.

The membrane can comprise a plurality of hollow fibers and the sample having a reduced content of water can then be contacted with at least one exterior wall of one of the hollow fibers. The hollow fiber membrane can comprise a cartridge comprising the hollow fibers. The cartridge can comprise an inlet for receiving the gas sample and an outlet for exiting the gas sample having a reduced content of water. The inlet and the outlet are in fluid flow communication with interior walls of the hollow fibers and disposed at each extremities of the hollow fibers. The cartridge can further comprise a purge inlet adapted to receive the gas sample having a reduced content of water. The gas purge inlet can be disposed downstream of the at least one gas sensor and being in fluid flow communication with the at least one gas sensor and with the exterior walls of the hollow fibers. The cartridge can also comprise a purge outlet which is in fluid flow communication with the exterior walls of the hollow fibers and the purge inlet, the purge outlet being adapted to exit water from the cartridge.

For example, the volume flow rate of the gas contacting the exterior wall of the at least one hollow fiber can be at least 2 times greater or about 2 to 3 times than the volume flow rate of the gas sample passed through the membrane so as to least partially reduce the amount of water present therein. For example, the volume flow rate of gas entering the purge inlet of the cartridge can be at least 2 times greater or about 2 to 3 times greater than the volume flow rate of gas entering the inlet of the cartridge.

The at least one gas sensor can be for example chosen from MOS (Metal Oxide Semiconductor) gas sensors, QMB (Quartz Microbalance) gas sensors, IRS (Infra-Red Sensor) gas sensors, CPS (Conducting Polymer Sensor) gas sensors, SAW (Surface Acoustic Wave) gas sensor, and OFS (Optical Fiber Sensor) gas sensors. For example, the at least one gas sensor can be a metal oxide semiconductor sensor.

In the method and apparatuses for detecting odor, the odor detection can be carried out for example in a continuous manner. The gas samples of a predetermined volume can be provided and analyzed in a continuous manner. For example, the method can be carried out in a continuous manner so as to analyze a plurality of gas samples one after the other, each gas sample of a predetermined volume being passed through the membrane so as to reduce the content of water present therein, contacted with the at least one gas sensor, and used to purge water out of the membrane. Alternatively, the method can be carried out in a non-continuous manner. Detection of the at least one odor can further comprise measuring the concentration of the at least one odor in the gas sample.

The means for at least partially reducing an amount of water present in the gas sample can comprise a membrane adapted to be at least substantially permeable to water and at least substantially impermeable to the at least one odor. The membrane can be a hollow fiber membrane comprising at least one hollow fiber into which the gas sample is passed through. The membrane can be a hollow fiber membrane comprising a plurality of hollow fibers.

The apparatuses can further comprise means for controlling the pressure of the gas sample. For example, the means for controlling the pressure of the gas sample can comprise a vacuum pump, a flow controller and a pressure gauge. The means for at least partially reducing the amount of water present in the gas sample can comprise a membrane adapted to be at least substantially permeable to water and at least substantially impermeable to the at least one odor. The hollow fiber membrane can comprise a cartridge comprising the hollow fibers. The cartridge can comprise an inlet for receiving the gas sample and an outlet for exiting the gas sample. The inlet and the outlet are in fluid flow communication with interior walls of the hollow fibers and disposed at each extremities of the hollow fibers. The outlet is in fluid flow communication with the at least one gas sensor. The cartridge can further comprise a gas purge inlet adapted to receive a purge gas. The gas purge inlet can be disposed downstream of the at least one gas sensor and being in fluid flow communication with the at least one gas sensor and with the exterior walls of the hollow fibers. The cartridge can also comprise a gas purge outlet which is in fluid flow communication with the exterior walls of the hollow fibers and the gas purge inlet. The gas purge outlet can be adapted to exit water from the cartridge. The apparatus can comprise a flow controller disposed between the at least one gas sensor and the gas purge inlet. The apparatus can comprise a vacuum pump disposed downstream of the gas purge outlet. The apparatus can also comprise a pressure gauge disposed between the vacuum pump and the gas purge outlet.

### BRIEF DESCRIPTION OF THE DRAWINGS

Further features and advantages will become more readily apparent from the following description of various embodiments as illustrated by way of examples in the appended drawings wherein:

FIG. 1 is a schematic representation of an apparatus for detecting and measuring odors according to an example;

FIG. 2 is a perspective view of an electronic nose as found in the prior art;

FIG. 3 is a bar chart showing that the resistance as a function of various examples of electronic nose used;

FIG. 4 is a graph representing the performance as a function of time of an example of membrane suitable for use in an apparatus for detecting and measuring odors, wherein humidity at the inlet of the membrane was maintained at 54%, a vacuum of 15 inches or mercury was used as well as a 3 lpm entry flow rate;

FIG. 5 is a graph representing the variation of humidity at the exit of an example of a membrane suitable for use in an apparatus for detecting and measuring odors, as a function of the flow rate of a purge gas entering into the membrane;

FIG. 6 is a graph representing the humidity at the exit of an example of a membrane suitable for use in an apparatus for detecting and/or measuring odors, as a function of the vacuum applied to a purge exit of the membrane, wherein the flow rate was maintained at 2.5 lpm in entry and the temperature at 29.3° C.;

FIG. 7 is a graph representing the dew point of a purge gas as a function of the nature of the membrane used and the flow rate of such a membrane, such membranes being examples of suitable membranes for use in an apparatus for detecting and measuring odors (inlet dew point of 20° C.);

FIG. 8 is a schematic detailed representation of the membrane used in the apparatus of FIG. 1, wherein the membrane is used in a backflow mode; the gas sample after passing through the interior of the membrane is used as a purge gas so as to cause water to exit the membrane; and

FIG. 9 is a schematic detailed representation of an example of a membrane that can be used in an apparatus for detecting odors, wherein a purge gas different that the gas sample passed though the membrane is used so as to cause water to exit the membrane.

### DETAILED DESCRIPTION OF VARIOUS EMBODIMENTS

The following examples represent in a non-limitative manner, various specific embodiments.

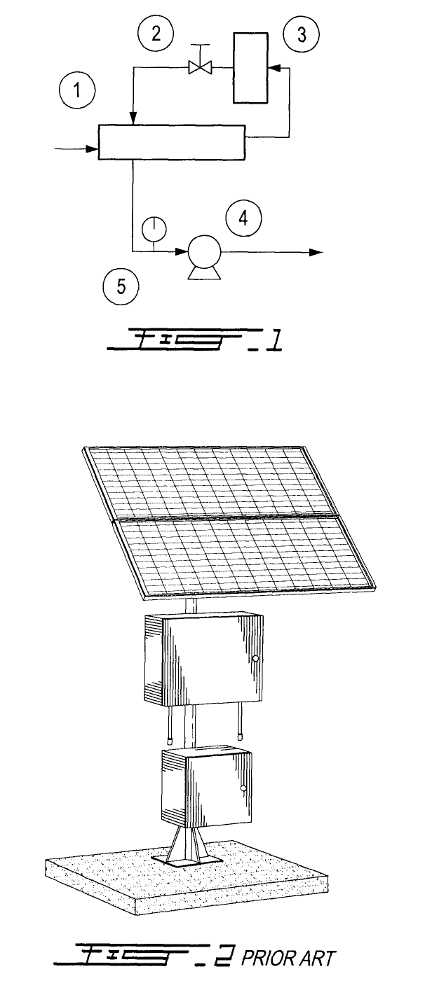

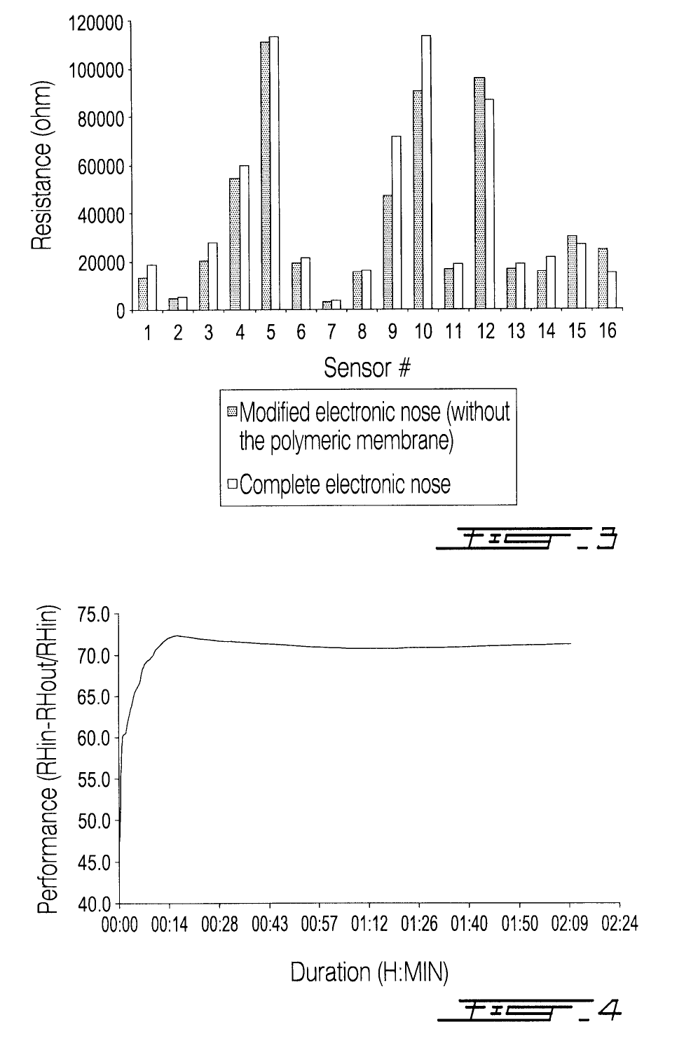

As it can be seen in FIG. 1, there is provided an apparatus for detecting and/or measuring odors. The apparatus comprises means for at least partially reducing an amount of water present in the gas sample. For example, such means can comprise a membrane 1 which is adapted to be at least substantially permeable to water and at least substantially impermeable to at least one odor. Such a membrane thus allows a substantial dehumidification of the gas sample while maintaining its content of odor i.e. the concentration of the at least one odor is not substantially affected by such a dehumidification treatment carried out by passing the gas sample through the membrane.

The membrane is in fluid flow communication with a measurement chamber 3 which comprises at least one gas sensor. The at least one gas sensor can be for example chosen from MOS (Metal Oxide Semiconductor) gas sensors, QMB (Quartz Microbalance) gas sensors, IRS (Infra-Red Sensor) gas sensors, CPS (Conducting Polymer Sensor) gas sensors, SAW (Surface Acoustic Wave) gas sensor, and OFS (Optical Fiber Sensor) gas sensors. According to a specific embodiment, the apparatus comprises a plurality of sensors. Each of the sensors can be adapted to detect and measure a particular odor. Each sensor is thus adapted to detect and measure several compounds associated to a particular odor. These sensors can be chosen from metal oxide semiconductor sensors.

The apparatus of FIG. 1 can further comprise means for controlling the pressure of the gas sample such as a vacuum pump, a flow controller and a pressure gauge.

The measurement chamber 3 is connected to and in fluid flow communication with a flow controller 2. Such a flow controller permits to control the backflow or purge gas which is introduced into the membrane 1 so as to cause water to exit from the membrane. According to another embodiment, the purge gas can be different than the dehumidified gas sample. The apparatus shown in FIG. 1 can also comprise, downstream of the membrane 1, a vacuum pump 4 and a pressure gauge 5.

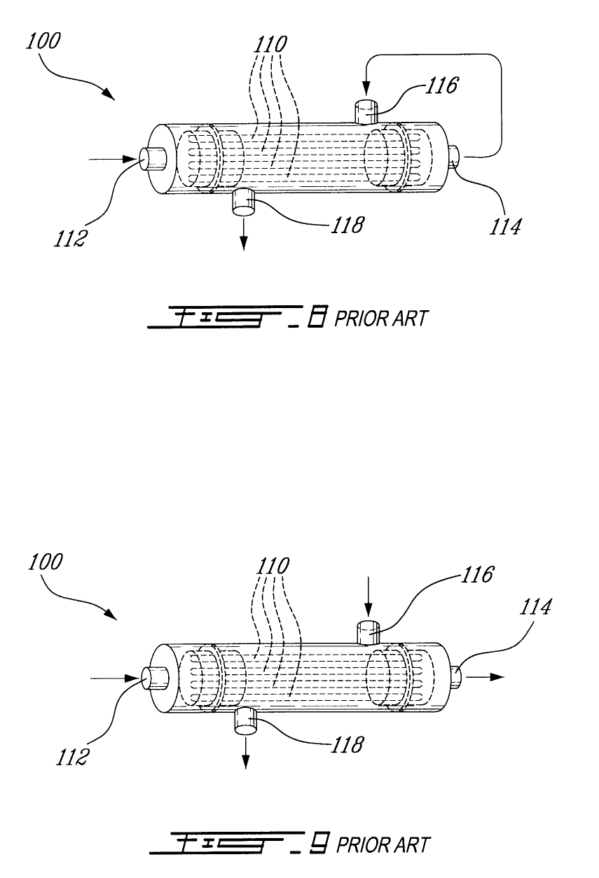

The membrane can be, for example, a hollow fiber membrane comprising a cartridge comprising hollow fibers. Such a membrane can be a membrane as shown in FIG. 8, which comprises a cartridge 100 including a plurality of hollow fibers 110. The cartridge 100 comprises an inlet 112 for receiving the gas sample and an outlet 114 for exiting the gas sample. The inlet 112 and the outlet 114 being in fluid flow communication with interior walls of the hollow fibers 110 and disposed at each extremities of the hollow fibers. For example, the membrane schematically represented in FIG. 8 can be a Perma Pure PD™-Series gas membrane. Such a membrane comprises hollow fibers made of Nafion™. As shown in Table 1, some tests have been made so as to determine the permeability of such a membrane.

TABLE 1
Chemical selectivity of the membrane

| Compound | Permeability |
|---|---|
| Water [H₂O] | Yes |
| n-butanol [CH₃(CH₂)₃OH] | Yes |
| Hydrogen sulphide [H₂S] | No |
| Methane [CH₄] | No |
| Carbon Dioxyde [CO₂] | No |

It was also observed that due to the chemical composition of the membrane used, the latter has a high selectivity (permeability) for water molecules, and more specifically for the presence of water vapor. Moreover, the membrane system is highly resistant to chemical attack, and therefore not corrodible. Chemical retention of the water molecules in vapor phase is thus effected before the measurement chamber.

The outlet 114 can is in fluid flow communication with the at least one gas sensor of the measurement chamber 3. The cartridge 100 further comprises a gas purge inlet 116 adapted to receive a purge gas, the gas purge inlet being disposed downstream of the at least one gas sensor of the measurement chamber 3 and being in fluid flow communication with the at least one gas sensor and with the exterior walls of the hollow fibers 110. The cartridge 100 also comprises a gas purge outlet 118 which is in fluid flow communication with the exterior walls of the hollow fibers 110 and the gas purge inlet 116. The gas purge outlet 118 is adapted to exit water from the cartridge 100.

As shown in FIG. 1, the purge gas used is the dehumidified gas sample. Alternatively, another gas can be used and introduced in the purge inlet 116. When using the apparatus of FIG. 1, the odor detection is carried out in a continuous manner. Gas samples of a predetermined volume are provided and analyzed in a continuous manner.

The gas sample to be analyzed is passed through the membrane 1 (see FIG. 1) and more particularly fed through the inlet 112 and passed through the hollow fibers 110 of the cartridge 100 when using such a membrane (see FIG. 8) so as to least partially reduce the amount of water present therein and to obtain a gas sample having a reduced content of water as compared to the gas sample before passing through the membrane. Then, the gas sample having a reduced content of water (or dehumidified gas sample) being exited by the outlet 114 is then contacted with the at least one gas sensor disposed within the measurement chamber 3 so as to detect the presence or absence of at least one odor. The concentration of the odor can also be measured. Then, the gas sample is introduced in the purge inlet 116 and then contacted with the exterior walls of the hollow fibers 110 so as to cause water to exit the cartridge 100. As previously indicated, such a method can be carried out in a continuous manner so as to analyze a plurality of gas samples one after the other, each gas sample of a predetermined volume being passed through the membrane so as to reduce the content of water present therein, contacted with the at least one gas sensor, and used to purge water out of the membrane. For example, the volume flow rate of gas entering in the purge inlet of the cartridge can be at least 2 times greater than the volume flow rate of gas entering the inlet of the cartridge. According to another embodiment, the volume flow rate of gas entering in the purge inlet of the cartridge can be about 2 to 3 times greater than the volume flow rate of gas entering the inlet of the cartridge.

As shown in FIG. 3, several tests have been made so as to demonstrate that the use of such a membrane does not affect the response of the sensor vis-à-vis the odor(s) contained in the gas sample. As it can be seen in FIG. 3, the tests made demonstrate the efficiency of the polymer membrane in the measurement of an odor. Efficiency refers to the rate of retention of, and selectivity for, water molecules. Table 2 and FIGS. 3 to 6 show the results obtained from a sample in which a biogas odor was presented to the electronic nose. The biogas contains primarily methane and carbon dioxide, the proportion of the latter two depending on the nature of the substrate. The tests made demonstrate that the membrane does not influence the response of the sensors when they are exposed to these biogas odors (concentration of the order of 2,200 o.u./m³).

TABLE 2
Performance tests carried out on the membrane.

| Humidity at inlet | Humidity at exit | Water fraction removed |
|---|---|---|
| (%) | (%) | (%) |
| 5.8 | 3.8 | 34.1 |
| 14.6 | 10.0 | 31.3 |
| 25 | 14.1 | 43.6 |
| 35 | 15.6 | 55.4 |
| 45.8 | 16.5 | 63.9 |
| 55 | 17.1 | 68.8 |
| 66.1 | 18.9 | 71.5 |
| 80.3 | 18.3 | 77.3 |

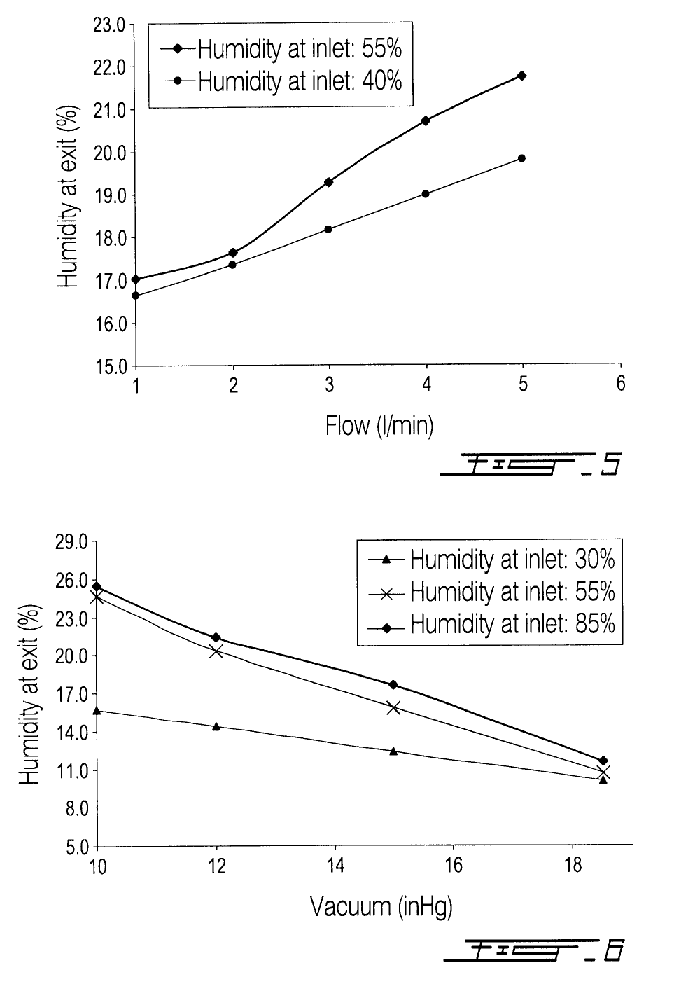

Moreover, the results prove that for given and maintained operating conditions (0.5 atm vacuum and 3 lpm exit flow rate, 29.0° temperature), the polymer membrane can maintain a practically constant humidity (see FIG. 4). At intake relative humidity levels close to saturation, relative humidity levels of less than 20% obtain at the membrane exit (see Table 2).

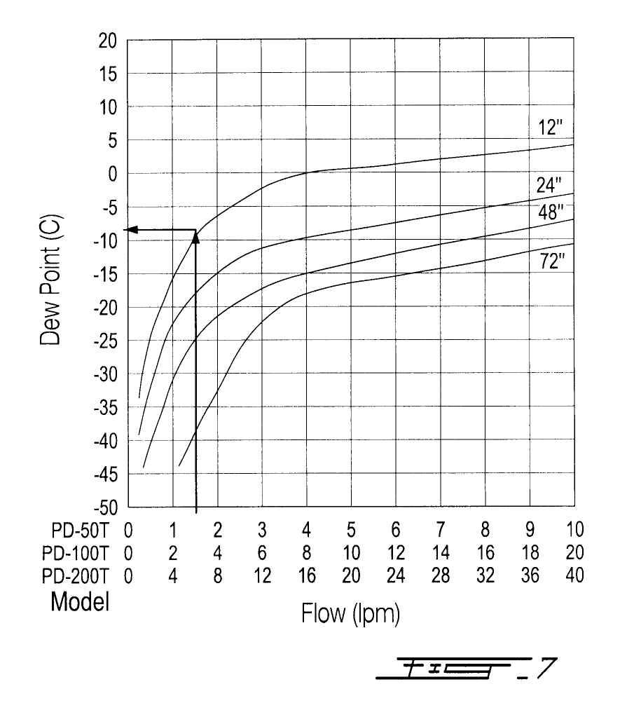

Membrane stability and response time were also evaluated. With stable inlet humidity, stable outlet humidity is obtained (a plateau is reached around 15% RH (removal of more than 60% of water) in a two-hour test) (see FIG. 4). The time required to reach that plateau (15% RH) was 27 minutes; the relative humidity was reduced by one-half in less than 5 minutes. The dependency of the membrane exit relative humidity on the flow rate and pressure was also evaluated (see FIGS. 5 and 6).

To reduce the water content of the gas sample, the apparatus needs some source of dry gas. There are various means for generating that dry air that can be used (air cylinder, zero air generator, filtration system, and others). The solution used in the case of FIGS. 8 and 9 was to operate the membrane in a backflow mode and to use the dehumidified gas sample so as to cause water to exit the membrane. The odorous air passing through the inner section of the tubular membrane (or through the interior wall of the hollow fibers) is then fed through the purge inlet so as to contact the exterior walls of the fibers thereby exiting water from the cartridge. This means that the odorous dehumidified air required for the operation of the membrane system comes from the humid odorous air after its passage through the membrane (through the fibers). In order to maintain the previously stated polymer membrane operating conditions, vacuum means, for example a vacuum of at least one-half-atmosphere will have to be maintained in the sampling system. A vacuum-type pump can be used. Such a pump can be, for example, a pump 12 Volts DC generating a maximum flow rate of 15.8 lpm and a maximum vacuum of 800 mbar. The vacuum control can be provided by a proportional controller connected to a solenoid valve. The backflow rate can be set physically by a capillary (or valve). The various elements of the apparatus (membrane, measuring chamber, flow controller, pressure gauge, and vacuum pump can be connected together by means of Teflon™ pipes.

The previously mentioned methods and apparatuses play a key role in odor measurement by means of electronic noses, regardless of the sensor technology used. Their great effectiveness was clearly demonstrated.

Since humidity plays an important role in sensor response, it was found that important advantages are obtained when using methods and apparatuses as previously defined. In fact, it was observed that when using such methods and apparatuses, it was possible to considerably reduce the undesirable effects of humidity on electronic noses and it was also possible to render them substantially independent of humidity having regard to their sensitivity and their analyses carried out on gas samples.

It was also found that the methods and apparatuses previously described allowed to reduce the relative humidity in the odorous sample while minimizing the alteration of the chemical composition of the odor. In other words, the chemical composition of the odorous sample on a dry basis was not altered and therefore, the same was observed concerning the response given by the gas sensor after analysis of the sample.

These methods and apparatuses thus have several advantages: 1) sensor response independent of humidity, 2) increased sensor sensitivity (the water molecules occupy fewer sites at the sensor surface), and 3) longer sensor service life.

### REFERENCES

1—S. Schiffman, H. T. Nagle, J. W. Gardner Handbook of Machine Olfaction: Electronic Nose Technology Editors: T. C. Pearce, S.
2—Norme européenne-Qualité de l'air Détermination de la concentration d'une odeur par olfactométrie dynamique Octobre 2003.
3—Compendium of Methods for the Determination of Toxic Organic Compounds in Ambient Air Second Edition Compendium Method TO-14A Determination Of Volatile Organic Compounds (VOCs) In Ambient Air Using Specially Prepared Canisters With Subsequent Analysis By Gas Chromatography Center for Environmental Research Information Office of Research and Development U.S. Environmental Protection Agency Cincinnati, Ohio January 1997.

While a description was made with particular reference to the illustrated embodiments, it will be understood that numerous modifications thereto will appear to those skilled in the art. Accordingly, the above description and accompanying drawings should be taken as specific examples and not in a limiting sense.

1. A method for detecting at least one odor in a gas sample, said method comprising:
at least partially reducing an amount of water present in said gas sample by means of a membrane adapted to be at least substantially permeable to water and at least substantially impermeable to said at least one odor; and
detecting the presence or absence of at least one odor in said sample.

2-12. (canceled)

13. The method of claim 1, wherein about 10% to about 75% of water present in said gas sample is removed.

14. The method of claim 1, wherein said odor detection is carried out in a continuous manner, in which gas samples of a predetermined volume are provided and analyzed in a continuous manner, or said odor detection is carried out in a non-continuous manner.

15. (canceled)

16. The method of claim 1, wherein said membrane is a hollow fiber membrane comprising at least one hollow fiber into which said gas sample is passed through.

17. The method of claim 16, wherein said gas sample is passed through said membrane so as to least partially reduce the amount of water present therein so as to obtain a gas sample having a reduced content of water as compared to said gas sample before passing through said membrane, said gas sample having a reduced content of water being then contacted with said at least one gas sensor so as to detect the presence or absence of at least one odor, and a purge gas is contacted with an exterior wall of said at least one hollow fiber so as to cause water to exit said membrane.

18. The method of claim 16, wherein said gas sample is passed through said membrane so as to least partially reduce the amount of water present therein so as to obtain a gas sample having a reduced content of water as compared to said gas sample before passing through said membrane, said gas sample having a reduced content of water being then contacted with at least one gas sensor so as to detect the presence or absence of said at least one odor, and said gas sample having a reduced content of water being then contacted with an exterior wall of said at least one hollow fiber so as to cause water to exit said membrane.

19. (canceled)

20. The method of claim 16, wherein said membrane comprises a plurality of hollow fibers and wherein said sample having a reduced content of water is then contacted with at least one exterior wall of one of said hollow fibers, and wherein said hollow fiber membrane comprises a cartridge comprising said hollow fibers, said cartridge comprising an inlet for receiving said gas sample and an outlet for exiting said gas sample having a reduced content of water, said inlet and said outlet being in fluid flow communication with interior walls of said hollow fibers and disposed at each extremities of said hollow fibers, said cartridge further comprising a purge inlet adapted to receive said gas sample having a reduced content of water, said gas purge inlet being disposed downstream of said at least one gas sensor and being in fluid flow communication with said at least one gas sensor and with the exterior walls of said hollow fibers, said cartridge also comprising a purge outlet which is in fluid flow communication with the exterior walls of said hollow fibers and said purge inlet, said purge outlet being adapted to exit water from said cartridge.

21. The method of claim 17, wherein said method is carried out in a continuous manner so as to analyze a plurality of gas samples one after the other, each gas sample of a predetermined volume being passed through said membrane so as to reduce the content of water present therein, contacted with said at least one gas sensor, and used to purge water out of said membrane, or said method is carried out in a non-continuous manner.

22-24. (canceled)

25. The method of claim 17, wherein said at least one gas sensor is a metal oxide semiconductor sensor.

26-29. (canceled)

30. The method of claim 17, wherein said at least one gas sensor is a metal oxide semiconductor sensor, a Quartz Microbalance Sensor, an Infra-Red Sensor, a Conducting Polymer Sensor, a Surface Acoustic Wave Sensor or an Optical Fiber Sensor.

31. The method of claim 1, wherein detection of said at least one odor further comprises measuring the concentration of said at least one odor in said gas sample.

32. A method for reducing losses of sensitivity of at least one gas sensor adapted to detect and/or measure at least one odor in a gas sample, said method comprising at least partially reducing an amount of water present in said gas sample by means of a membrane adapted to be at least substantially permeable to water and at least substantially impermeable to said at least one odor, before contacting said sample with said at least one sensor so as to detect and/or measure said at least one odor in said gas sample.

33-49. (canceled)

50. The method of claim 32, wherein about 10 to about 75% of water present in said gas sample is removed.

51. (canceled)

52. The method of claim 32, wherein said membrane is a hollow fiber membrane comprising at least one hollow fiber into which said gas sample is passed through.

53-64. (canceled)

65. An apparatus for detecting and/or measuring odors in a gas sample, said apparatus comprising:
means for at least partially reducing an amount of water present in said gas sample; and
at least one gas sensor adapted to detect and/or measure odors, said at least one gas sensor being in fluid flow communication with said means for at least partially reducing an amount of water present in said gas sample and being disposed downstream of the latter,
wherein said means for at least partially reducing the amount of water present in said gas sample comprises a membrane adapted to be at least substantially permeable to water and at least substantially impermeable to said at least one odor.

66. (canceled)

67. The apparatus of claim 66, wherein said apparatus further comprises means for controlling the pressure of said gas sample and wherein said means for controlling the pressure of said gas sample comprises a vacuum pump, a flow controller and a pressure gauge.

68. (canceled)

69. The apparatus of claim 65, wherein said membrane is a hollow fiber membrane comprising at least one hollow fiber into which said gas sample is passed through.

70. (canceled)

71. The apparatus of claim 65, wherein said membrane is a hollow fiber membrane comprising a plurality of hollow fibers into which said gas sample is passed through, wherein said hollow fiber membrane comprises a cartridge comprising said hollow fibers, said cartridge comprising an inlet for receiving said gas sample and an outlet for exiting said gas sample, said inlet and said outlet being in fluid flow communication with interior walls of said hollow fibers and disposed at each extremities of said hollow fibers, said outlet being in fluid flow communication with said at least one gas sensor, said cartridge further comprising a gas purge inlet adapted to receive a purge gas, said gas purge inlet being disposed downstream of said at least one gas sensor and being in fluid flow communication with said at least one gas sensor and with the exterior walls of said hollow fibers, said cartridge also comprising a gas purge outlet which is in fluid flow communication with the exterior walls of said hollow fibers and said gas purge inlet, said gas purge outlet being adapted to exit water from said cartridge.

72. The apparatus of claim 71, wherein said apparatus comprises a flow controller disposed between said at least one gas sensor and said gas purge inlet.

73. The apparatus of claim 71, wherein said apparatus comprises a vacuum pump disposed downstream of said gas purge outlet.

74. The apparatus of claim 73, wherein said apparatus comprises a pressure gauge disposed between said vacuum pump and said gas purge outlet.
